(19) United States
(12) Patent Application Publication
(10) Pub. No.: US 2017/0368176 A1
(43) Pub. Date: Dec. 28, 2017

(54) NON-TOXIC TOPICAL ANESTHETIC OPHTHALMIC COMPOSITIONS

(71) Applicant: The Children's Medical Center Corporation, Boston, MA (US)

(72) Inventors: Daniel S. Kohane, Newton, MA (US); James B. McAlvin, Walpole, MA (US); Changyou Zhan, Jamaica Plain, MA (US)

(21) Appl. No.: 15/536,286

(22) PCT Filed: Dec. 14, 2015
(86) PCT No.: PCT/US15/65554

§ 371 Date: Jun. 15, 2017

Related U.S. Application Data
(60) Provisional application No. 62/171,611, filed on Jun. 5, 2015.
(60) Provisional application No. 62/092,078, filed on Dec. 15, 2014.

(51) Int. Cl. A61K 45/06; A61K 9/00; A61K 31/519; A61K 47/12

(52) U.S. Cl. CPC A61K 45/06; A61K 47/12; A61K 9/0048; A61K 31/519; A61K 2300/00

(57) ABSTRACT

Compositions and methods for prolonging the local anesthetic effect of site 1 sodium channel blockers and local anesthetics with minimal or reduced toxicity have been developed for ophthalmic use. It has been established that agents such as dexmedetomidine having alpha-2-adrenergic agonist and Hyperpolarization-activated cyclic nucleotide-gated channel antagonist activity can dramatically prolong the duration of nerve blockade when administered to the surface of, a compartment of or tissue adjacent to, the eye. A preferred active agent for prolonging the local anesthetic effect of site 1 sodium channel blockers or local anesthetics is Dexmedetomidine.

### CROSS REFERENCE TO RELATED APPLICATIONS

This application claims priority to and benefit of U.S. Provisional Application No. 62/092,078 “Compositions and Methods for Prolonging Local Anesthesia” filed on Dec. 15, 2014, and U.S. Provisional Application No. 62/171,611 “Non-Toxic Topical Anesthetic Ophthalmic Compositions” filed Jun. 5, 2015, the disclosures of which are hereby incorporated herein by reference in their entirety.

### STATEMENT REGARDING FEDERALLY SPONSORED RESEARCH OR DEVELOPMENT

This invention was made with government support under grant no. NIGMS GM073626 awarded by the National Institutes of Health. The government has certain rights in the invention.

### FIELD OF THE INVENTION

The present disclosure relates generally to the field of improved nerve blocks and infiltration local anesthesia and analgesia with no increase in toxicity, especially for application to the cornea.

### BACKGROUND OF THE INVENTION

Conventional amino-ester and amino-amide local anesthetics are used to reduce ocular pain related to corneal injury and ophthalmic surgery. They act by binding to an intracellular domain of the sodium channel and blocking sodium influx. They produce corneal anesthesia for 15-20 minutes when applied topically, with return of normal sensation after 60 min, and so require repeated administration. Brief ophthalmic procedures, such as cataract extraction, are routinely performed under local anesthesia with conventional local anesthetics, which are administered by a variety of techniques, including topical application onto the cornea, injection into or around the muscle cone, and injection under the Tenon's capsule. Given frequently, these agents may delay epithelial healing, cause anterior segment inflammation, corneal ulceration, and occasionally neurotrophic keratopathy. Their short durations of action and the potential for tissue toxicity exclude their use in lengthier ophthalmic procedures, and limit their use for other causes of corneal pain such as traumatic abrasions and recurrent erosions.

An ocular anesthetic formulation with prolonged effect and minimal toxicity is needed. Such a formulation could be used to prevent pain more effectively during longer surgical procedures and for outpatient management of minor corneal injury during the period when ocular pain is most intense. Addition of the α₂-adrenergic receptor (α₂-AR) agonists clonidine or dexmedetomidine to conventional amino-amide or amino-ester local anesthetics extends the duration of peripheral nerve block from infiltration anesthesia, including ocular anesthesia via sub-Tenon's injection (i.e. peripheral nerve blockade of distal branches of the ophthalmic branch of the trigeminal nerve by episcleral; placement of a catheter under the sclera via small incision and threading of the catheter under the conjunctiva and on top of the sclera to the back of the globe for injection the anesthetic solution. In both of those cases, the conventional local anesthetic is believed to be toxic to the cornea, which could limit usefulness.

The prolonged duration local anesthetic EXPAREL®, an injectable liposomal formulation of bupivacaine, an amide local anesthetic, indicated for single-dose infiltration into the surgical site to produce postsurgical analgesia, provides unpredictable nerve blockade in humans that peaks at 24 h after injection and the anesthetic effect is inversely proportional to dose. Moreover it entails the use of a sustained release system and causes local tissue injury and inflammation. Similar considerations relate to bupivacaine and dexamethasone microparticles, which could provide prolonged duration local anesthesia albeit with a sustained release system and with severe tissue injury.

The quaternary lidocaine derivative QX-314 could provide prolonged duration local anesthesia (approximately 24 h duration) but has severe local tissue injury and systemic toxicity. When amino-amide and amino-ester local anesthetics are given in overdose or via inadvertent intravascular injection, they generate cardiovascular toxicity that is notoriously refractory to resuscitation (Polaner et al. Ped Aries 2011; 21:737-742; Fisher, et al., Can. J. Anesth., 1997; 44: 592-598; Butterworth, Reg. Anesth. Pain Med., 2010; 35:167-76). Bupivacaine cardiovascular toxicity is likely mediated by the cardiac sodium channel Nav1.5 which is relatively more resistant to binding and inactivation by site 1 sodium channel blockers (Clarkson, et al., Anesthesiology, 1985; 62:396-405).

Conventional local anesthetics are associated with local neurotoxicity in clinical doses and profound cardiovascular toxicity in overdose. While overall incidence is low, studies have also identified prolonged numbness and paresthesias as a complication of local and regional anesthesia with amide anesthetics. This has been associated with histological signs of chemical nerve injury (Myers, et al., Anesthesiology, 1986; 64:29-35; Kalichman, et al., J. Pharm. Exper. Therapeutics, 1989; 250(1):406-413). These risks for local neurotoxicity are likely to be further increased in the setting where prolonged pain relief is attempted via administration of conventional local anesthetics by controlled release delivery (Padera, et al., Anesthesiology, 2008; 108: 921-8; Kohane and Langer, Chem. Sci., 2010; 1: 441-446) or local perineural infusions, particularly when higher concentrations or doses are used for longer periods of time. In equipotent intrathecally injected doses, site 1 sodium channel blockers cause longer duration of anesthesia with less histologic evidence of neurotoxicity compared to bupivacaine (Sakura, et al., Anesth. Analg., 1995; 81:338-46). Overall, approaches to prolonged local anesthesia involving site 1 sodium channel blockers lower the risks of nerve injury compared to approaches involving prolonged or repeated administration of conventional amino-amides or amino-esters.

As described in U.S. Pat. No. 6,326,020 by Kohane, et al., combinations of naturally occurring site 1 sodium channel blockers, such as tetrodotoxin (TTX), saxitoxin (STX), decarbamoyl saxitoxin, and neosaxitoxin, with other agents, have been developed to give long duration block with improved features, including safety and specificity. In one embodiment, duration of block is greatly prolonged by combining a toxin with a local anesthetic, vasoconstrictor, glucocorticoid, and/or adrenergic drugs, either alpha agonists (epinephrine, phenylephrine) or mixed central-peripheral alpha-2 agonists (clonidine), or other agents. Prolonged nerve block can be obtained using combinations of toxin with vanilloids. Dosage ranges based on studies with tetrodotoxin and saxitoxin were provided. However, it is now known that studies must be conducted with each toxin in order to predict the effective dosages, since dosages with one type of toxin are not predictive of efficacy with another type of toxin. It has also been discovered that one cannot extrapolate from rats or sheep to humans to determine safe and efficacious dosages with respect to these toxins. As demonstrated below, many of the considerations relating to peripheral nerve blockade are not applicable to formulations administered to the eye.

It is therefore an object of this invention to provide ophthalmic compositions and methods for prolonging local anesthesia of the eye in the absence of toxic side effects.

It is a further object of the present invention to provide specific combinations of two or more different classes of drugs which are both safe and efficacious in humans, which elicit more prolonged ocular nerve blockade for up to two to three days following a single application, with no to minimal toxicity, either to the corneal epithelium or systemically.

### SUMMARY OF THE INVENTION

It has been established that the duration of local anesthesia to the eye induced by site 1 sodium channel blockers can be prolonged by combining with additional active agents such as alpha-2-adrenergic agonists and/or Hyperpolarization-activated cyclic nucleotide-gated channel antagonists with limited or reduced toxicity relative to the site 1 sodium channel blockers alone, especially as applied to the eye. Tetrodotoxin (TTX), saxitoxin (STX), and neosaxitoxin (NeoSTX) are preferred examples of potent local anesthetics that act by binding to site 1 on the extracellular part of the sodium channel and blocking sodium influx. S1SCBs produce corneal analgesia with minimal toxicity to the corneal epithelium.

In the preferred embodiment, the agent in combination with site 1 sodium channel blocker is dexmedetomidine, which has been discovered to have significant and unprecedented benefits, unlike other alpha-2 adrenergic agonists, in combination with a site 1 sodium channel blocker, especially when applied to the cornea, particularly as compared to the same combination applied to peripheral nerves or combinations in which another alpha-2 adrenergic agonist such as clonidine is used. Similar results were obtained with local anesthetics in combination with dexmedetomidine.

Pharmaceutical compositions including one or more site 1 sodium channel blockers and one or more alpha-2-adrenergic agonists and/or hyperpolarization-activated cyclic nucleotide-gated channel antagonists in an amount effective to reduce, decrease, or inhibit sensory and/or motor function in the cornea of a subject compared to a control are described. A preferred alpha-2-adrenergic agonist is dexmedetomidine, or an analog or functional variant thereof, in a concentration of dexmedetomidine between 0.21 mM and 1.1 mM, inclusive. In certain embodiments, the amount of one or more site 1 sodium channel blockers and dexmedetomidine is effective to reduce, decrease, or inhibit sensory and/or motor function in the cornea of the subject without reducing, decreasing, or inhibiting sensory and/or motor function outside of the area of the cornea, for example, in an amount that does not induce sedation.

Pharmaceutical compositions including a delivery vehicle are also disclosed. In one embodiment, the delivery vehicle is a liposome. Exemplary liposomes comprise 1,2-dioleoyl-sn-glycero-3-phosphocholine, 1,2-dioleoyl-sn-glycero-3-phospho-(1′-rac-glycerol) and cholesterol. In a particular embodiment, liposomes include a molar ratio of 1,2-dioleoyl-sn-glycero-3-phosphocholine to 1,2-dioleoyl-sn-glycero-3-phospho-(1′-rac-glycerol) of about 15:1 and a molar ratio of total lipid to cholesterol of about 4:2.

As described in the examples, solutions of TTX±dexmedetomidine, TTX±clonidine, STX±dexmedetomidine, dexmedetomidine or proparacaine were applied to the rat cornea. Tactile sensitivity was measured by recording the blink response to probing of the cornea with a Cochet-Bonnet esthesiometer. The duration of corneal anesthesia was calculated. Cytotoxicity from anesthetic solutions was measured in vitro. The effect on corneal healing was measured in vivo after corneal debridement followed by repeated drug administration. Addition of dexmedetomidine to TTX or STX significantly prolonged corneal anesthesia beyond that of either drug alone, whereas clonidine did not. TTX or STX co-administered with dexmedetomidine resulted in 2-3 times longer corneal anesthesia than did proparacaine. S1SCB-dexmedetomidine formulations were not cytotoxic nor was corneal healing delayed significantly by any of the test solutions.

Co-administration of S1SCBs and local anesthetic with dexmedetomidine provided prolonged corneal anesthesia without delaying corneal wound healing. Such formulations should be useful for the management of acute surgical and non-surgical corneal pain. Co-administration of local anesthetics with adjuvant agents can enhance anesthetic effect and/or (in the case of S1SCBs) prevent potential systemic toxicity by reducing the dose required for a given effect.

### BRIEF DESCRIPTION OF THE DRAWINGS

FIG. 1 is a line graph showing % cell survival throughout the incubation time (h) for citrate buffer; 0.21 mM DMED; 3.1 mM TTX; and 3.1 mM TTX+0.21 mM DMED, respectively. n=4. All p values were >0.05.

FIG. 2 is a histogram showing the rate of cornea re-epithelialization (mm²/h) in samples receiving 0.9% (weight/volume) saline; 15 mM PPC; 3.1 mM TTX; 3.1 mM TTX+0.21 mM DMEM; and 0.21 mM DMEM, respectively. Rate of re-epithelialization was averaged 20 hours after surgical debridement of a 3 mm2 area of corneal epithelium (n=5, p=0.320). For both Figs, TTX=Tetrodotoxin; DMED=Dexmedetomidine. Data are mean±SD.

FIG. 3 is a graph showing the Duration of Block<6 (i.e., time without blink response to a 6 cm filament) in minutes as a function of the rate of corneal re-epithelialization (mm²/h) after topical application of 3.1 mM Tetrodotoxin (TTX); 0.21 mM Dexmedetomidine (DMED); 15 mM Proparacaine (PPC) or saline, respectively. n=18 for 3.1 mM TTX+0.21 mM DMED. n=5 for all re-epithelialization groups. n=14 for 3.1 mM TTX. n=10 for 15 mM PPC. n=5 for saline and 0.21 mM DMED. Data are medians with interquartile ranges for corneal block durations and means±SD for corneal re-epithelialization rates.

FIG. 4 is a graph of the dose response curve of sedation score over time (min) for DMED showing that increasing doses of DMED do not increase block further but sedation results from the higher two doses. All are 3.1 mM TTX. Diamonds, 1.1 mM (5.3-5.7 μg/kg DMED); inverted triangle, 0.21 mM (4.6-5.0 μg/kg DMED); triangle, 0.53 mM (11.9-12.7 μg/kg DMED); and circle, 1.1 mM (23.4-25 μg/kg DMED).

FIGS. 5A-5D are graphs of pain blockade (filament length) over time (minutes) for liposomal encapsulated anesthetic formulation, Lipid A, Lipid B, Lid C, and Lipid D, as a function of the size of the liposome (120, 450, and 850 nm). FIGS. 5A and 5C demonstrate the effects of TTX/DMED solution and liposomes without dialysis (with unencapsulated free drug in the formulations) on ocular anesthesia. FIGS. 5B and 5D display the effects of TTX/DMED solution and liposomes after dialysis (without unencapsulated free drug in the formulations) on ocular anesthesia.

### DETAILED DESCRIPTION OF THE INVENTION

The safety benefits of reducing anesthetic dosing and toxicity are important for patients at all ages. It has been established that the duration of local anesthesia by site 1 sodium channel blockers (S1SCB) can be prolonged by combining these agents with other active agents, such as alpha-2-adrenergic agonists and/or hyperpolarization-activated cyclic nucleotide-gated channel antagonists. Formulations include one or more agents that block site 1 sodium channels, in combination with one or more agents that act as alpha-2-adrenergic agonists, in particular, dexmedetomidine (DMED). The combination of site 1 sodium channel blockers and alpha-2-adrenergic agonists prolong the duration of local anesthesia with limited or reduced toxicity relative to the site 1 sodium channel blockers alone. For example, DMED prolongs the duration of block by tetrodotoxin (TTX) in a dose dependent fashion (i.e., as the concentration of dexmedetomidine is increased, the duration of block from TTX increases).

Significant dissimilarities between the pharmacology of dexmedetomidine on the cornea and at the sciatic nerve have been identified. The mechanisms by which dexmedetomidine and clonidine prolong local anesthesia at non-corneal, peripheral nerves have been attributed to (1) vasoconstriction from α2-AR agonism and (2) blockade of the hyperpolarization cation current (Ih). It has been established that dexmedetomidine's effect on block from site 1 sodium channel blockers in the cornea may occur via a mechanism that is distinct. Compositions and methods for prolonging and enhancing the local analgesic effect of site 1 sodium channel blockers in the cornea have been developed.

### I. Definitions

The term “Analgesia” refers to insensibility to pain without loss of consciousness.

The term “Anesthetic” refers to a loss of sensation (local; not causing loss of consciousness; systemic, with loss of consciousness) and usually of consciousness without loss of vital functions.

The term “Anesthetic Adjuvant” refers to an active agent that enhances or prolongs the analgesic or anesthetic effect of an analgesic or anesthetic agent. Typically, the Anesthetic Adjuvant administered in combination with the analgesic or anesthetic agent produces an effect that is greater than the additive effect of either of the agents administered alone. Exemplary Anesthetic Adjuvants include alpha-2-adrenergic agonists; hyperpolarization-activated cyclic nucleotide-gated channel antagonists; and compounds that act as both alpha-2-adrenergic agonists and hyperpolarization-activated cyclic nucleotide-gated channel antagonists.

The term “Vasoconstrictor” is an agent narrowing of the lumen of blood vessels, especially as a result of vasomotor action.

The term “Infiltration” refers to injection into multiple layers or areas of tissue.

The term “Injection” refers to injection into a single point in tissue or lumen.

The term “Nerve block” refers to local anesthesia produced by interruption of the flow of impulses along a nerve trunk.

The term “Minimum effective concentration” or “MEC” is the lowest local concentration of one or more drugs in a given location sufficient to provide pain relief.

The phrase “pharmaceutically acceptable” refers to compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” refers to pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “pharmaceutically acceptable salts” is art-recognized, and includes relatively non-toxic, inorganic and organic acid addition salts of compounds. Examples of pharmaceutically acceptable salts include those derived from mineral acids, such as hydrochloric acid and sulfuric acid, and those derived from organic acids, such as ethanesulfonic acid, benzenesulfonic acid, and p-toluenesulfonic acid. Examples of suitable inorganic bases for the formation of salts include the hydroxides, carbonates, and bicarbonates of ammonia, sodium, lithium, potassium, calcium, magnesium, aluminum, and zinc. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine.

The phrase “prolonged residence time” is art-recognized and refers to an increase in the time required for an agent to be cleared from a patient's body, or organ or tissue of that patient.

The phrase “therapeutically effective amount” refers to an amount of the therapeutic agent that, when incorporated into and/or onto particles described herein, produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The terms “incorporated” and “encapsulated” refers to incorporating, formulating, or otherwise including an active agent into and/or onto a composition that allows for release, such as sustained release, of such agent in the desired application. The terms contemplate any manner by which a therapeutic agent or other material is incorporated into a polymer or liposomal matrix, including for example: attached to a monomer of such polymer (by covalent, ionic, or other binding interaction), physical admixture, enveloping the agent in a coating layer of polymer or lipid, and having such monomer be part of the polymerization to give a polymeric formulation, distributed throughout the polymeric or lipid matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition. More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer that it is dispersed as small droplets, rather than being dissolved, in the polymer.

### II. Combinations for Prolonged Local Anesthesia

Formulations for the prolonged duration of local anesthesia combine two or more classes of drugs, which act together to elicit prolonged sensory blockade of nerves around which they are delivered: site 1 sodium channel blockers and alpha-2-adrenergic agonists and/or blocks hyperpolarization-activated nonselective cation channels (HCN channels) blockers. The formulations can prolong the duration of local anesthesia as compared to administration of the active agents alone. Preferably, the formulations prolong the duration of local anesthesia when applied to the eye with limited or reduced toxicity.

Controlled release formulations including two or more classes of drugs which elicit prolonged sensory blockade for extended periods of time are also disclosed. The controlled release formulations can prolong the duration of local anesthesia and reduce or inhibit toxicity or other side effects associated with the active agents. Examples of controlled release formulations include nanoparticles and liposomes.

A. Site 1 Sodium Channel Blockers

Site 1 Sodium Channel blockers (S1SCB) are a family of molecules long recognized for their potent and specific blockade of voltage gated sodium channels. Site I sodium channel blockers bind to what is known as site 1 of the fast voltage-gated sodium channel Site 1 is located at the extracellular pore opening of the ion channel. The binding of any molecules to this site will temporarily disable the function of the ion channel. S1SCBs include phycotoxins (saxitoxin (STX), decarbamoyl saxitoxin, neosaxitoxin, and the gonyautoxins), tetrodotoxin (TTX) and several of the conotoxins.

A number of naturally occurring toxins have much greater intrinsic potency, concentrations in the 10⁻⁷ to 10⁻⁸M range, required to block conduction of nerve impulses. Tetrodotoxin systemic toxicity, like that of other local anesthetics, can result in diaphragmatic paralysis leading to respiratory arrest and death. Hypotension, presumably due to smooth muscle relaxation and/or vasomotor nerve blockade, is also a prominent feature. Tetrodotoxin is safer than convention local anesthetics in a hospital setting with the availability of respiratory support, in that cardiotoxicity is relatively minimal, and tetrodotoxin does not cause seizures. Clinically, the toxic syndrome is similar to curare poisoning.

In the mid-1970s, Adams, et al. reported that toxins such as tetrodotoxin and saxitoxin could be combined with conventional local anesthetics to prolong local anesthesia. See U.S. Pat. Nos. 3,966,934, 3,957,996, 4,001,413, 4,029,794, 4,029,793, and 4,022,899 to Adams, et al. Better results were obtained with inclusion of epinephrine. This technology was never developed clinically, however. Published data did not clearly demonstrate nociceptive block [loss of pain sensation]. Blockade was simply defined as loss of motor function in the injected limb. The possibility that systemic toxicity was the cause of the observed nerve blocks was also not assessed. The addition of a vasoconstrictor to slow systemic absorption was shown to reduce toxicity and decrease mortality, but neither effect was quantified.

1. Tetrodotoxin

Tetrodotoxin (TTX) is a highly potent neurotoxin that blocks the fast Na+ current in human myocytes (the contractile cells of the muscles), thereby inhibiting their contraction. Chemically, it is an amino perhydroquinoline (see Pharmacological Reviews, 18(2), 997-1049, 1966). Combinations of tetrodotoxin with bupivacaine produced long duration sciatic nerve blockade in rats without increased systemic toxicity compared to tetrodotoxin alone (Kohane, et al., Anesthesiology, 89:119-131, 1998). Although the potent inhibition of voltage-gated Na+ channels is too hazardous for TTX to be used as a drug alone, blocking such channels in a controlled fashion maybe desirable in the treatment of conditions such as Parkinson's disease and chronic pain in terminally ill cancer patients.

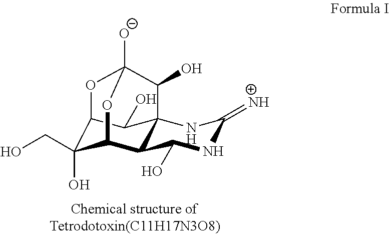

Tetrodotoxin has been isolated from animals, such as the blue-ringed octopuses, and is produced by bacteria. The most common bacteria associated with TTX production are Vibrio Sp. bacteria, with Vibrio-alginolyticus being the most common species. Pufferfish, chaetognaths, and nemerteans have been shown to contain Vibrio alginolyticus and TTX, however the link between these facts and production of TTX in animals has not been firmly established, and there remains much debate in the literature as to whether the bacteria are truly the source of TTX in animals. Although tetrodotoxin is perhaps the most widely known site 1 toxin, it is expensive for clinical use since it must come from the puffer fish. When the endo-symbiotic bacteria that makes TTX is grown ex vivo, its production of TTX diminishes. Tetrodotoxins can be obtained from the ovaries and eggs of several species of puffer fish and certain species of California newts. Numerous schemes for the total chemical synthesis of Tetrodotoxin has also been reported, including by Diels-Alder Reactions or Syntheses of TTX from Carbohydrates and Congeners (Ohyabu, et al., J Am Chem Soc. 23; 125(29): 8798-805 (2003)); Nishikawa, et al., Angew. Chem. Int. Ed., 43, 4782 (2004); reviewed in Chau and Ciufolini, Mar Drugs, 9(10): 2046-2074 (2011)). Synthesis features rapid construction of the cyclohexene by Diels-Alder cycloaddition using an enantiomerically-pure dienopile, the early introduction of the aminated quaternary center, and the use of that center to direct the relative configuration of further functionalization around the ring.

2. Phycotoxins

Phycotoxins act as a specific blocker of the voltage-dependent sodium channels present in excitable cells (Kao, C. Y., Pharm. Rev., 18: 997-1049 (1966)). Due to the inhibition of sodium channels, the transmission of a nervous impulse is blocked and the release of neurotransmitters is prevented at the level of the neuromotor junction, which prevents muscular contraction. Due to these physiological effects, these compounds are potentially useful in pharmacology when used as muscular activity inhibitors in pathologies associated with muscular hyperactivity, such as muscular spasms and focal dystonias, when applied locally in injectable form.

Additionally, since a blockage of the nervous impulse at the transmission level is generated when these compounds are applied as a local infiltration, they are not only able to block the efferent neurotransmission pathways, but also block afferent pathways and cause an inhibition of the sensory pathways and generate an anesthetic effect when these compounds are locally injected. Both effects are simultaneous, as described in U.S. Pat. No. 4,001,413.

The chemical structure of these phycotoxins has a general structure of Formula II:

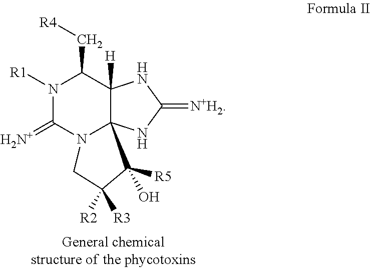

The particular chemical structure of the structure is defined by the substituents R1 to R5 according to the Table 1.

TABLE 1
Chemical Structures of Phycotoxins relative to the Formula I

| Compound | R1 | R2 | R3 | R4 | R5 |
|---|---|---|---|---|---|
| Saxitoxin | H | H | H | COONH₂ | OH |
| Neosaxitoxin | OH | H | H | COONH₂ | OH |
| Gonyaulatoxin 1 | OH | H | OSO⁻₃ | COONH₂ | OH |
| Gonyaulatoxin 2 | H | H | OSO⁻₃ | COONH₂ | OH |
| Gonyaulatoxin 3 | OH | OSO⁻₃ | H | COONH₂ | OH |
| Gonyaulatoxin 4 | H | OSO⁻₃ | H | COONH₂ | OH |
| Gonyaulatoxin 5 | H | H | H | COONHSO⁻³ | OH |

Saxitoxin

Saxitoxin (STX) was first extracted from the Alaska butterclam, Saxidomus gigantcus, where it is present in algae of the genus Gonyaulax. The reported molecular formula is C₁₀H₁₇N₇O4. It is freely soluble in water and methanol and it is believed the toxin has a perhydropurine nucleus in which are incorporated two guanidinium moieties. STX is responsible for paralytic shellfish poisoning. It is reported to be one of the most toxic non-protein compounds known, with a toxicity of 8 μg/Kg in mice (approximately 0.2-1.0 mg would prove fatal to humans), and is therefore widely considered too toxic to be used alone as a local anesthetic.

Neosaxitoxin and Decarbamoyl Saxitoxin

Two saxitoxin derivatives, neosaxitoxin (NeoSTX) and decarbamoyl saxitoxin, have advantages in terms of the production process and potency. A study examined rat sciatic nerve blockade with several members of the saxitoxin series, including NeoSTX (Kohane, et al., Reg. Anesth. Pain Med., 25:52-9 (2000). Saxitoxin and these two derivatives all give markedly synergistic block and prolonged block (1-2 days in rat sciatic nerve in vivo) when combined with bupivacaine or epinephrine.

Neosaxitoxin and decarbamoyl saxitoxin are potentially more potent and may have advantages over saxitoxin in formulation. Neosaxitoxin (NeoSTX) is under clinical development as a prolonged duration local anesthetic (Rodriguez-Navarro, et al., Anesthesiology, 106:339-45, 2007; Rodriguez-Navarro, et al., Neurotox. Res., 16:408-15, 2009; Rodriguez-Navarro, et al., Reg. Anesth. Pain Med., 36:103-9, 2011). A Phase 1 study of subcutaneous infiltration in human volunteers showed that NeoSTX caused effective cutaneous hypoesthesia (Rodriguez-Navarro, et al., Anesthesiology, 106:339-45, 2007) and a second Phase 1 study showed that combination with bupivacaine resulted in more prolonged analgesia compared to NeoSTX or bupivacaine alone (Rodriguez-Navarro, et al., Neurotox. Res., 16:408-15, 2009). Two U.S. patents recently issued on specific dosage formulations for neosaxitoxin, U.S. Pat. Nos. 8,975,281 and 8,975,268.

Saxitoxin was first extracted from the Alaska butterclam, Saxidomus gigantcus, where it is present in algae of the genus Gonyaulax. The reported chemical formula is C₁₀H₁₅N₇O₃.2HCl. It is believed the toxin has a perhydropurine nucleus in which are incorporated two guanidinium moieties. Saxitoxin is also too toxic to be used alone as a local anesthetic. Saxitoxin (STX) and its derivatives can be produced in bioreactors from algae. The phycotoxins neosaxitoxin, saxitoxin and gonyaulatoxins are active compounds produced by harmful algae blooms of the genera Alexandrium sp., Piridinium sp., and Gimnodinium sp., (Lagos, N. Biol. Res., 31: 375-386, 1998)). In the last 15 years, it has been demonstrated that these phycotoxins can also be produced by fresh water cyanobacteria such as photosynthetic blue-green algae, besides being produced by marine dinoflagellates.

Four genera of cyanobacteria able to produce paralyzing phycotoxins have been identified, and each produces a different mixture of phycotoxins both in amounts and in types of phycotoxins produced, i.e. they produce different profiles of paralyzing phycotoxins (Lagos, et al., 1999, TOXICON, 37: 1359-1373 (1999). Pereira, et al., TOXICON, 38: 1689-1702 (2000)). STX can also be produced by chemical synthesis according to at least three distinct methods (Kishi, et al., J. Am. Chem. Soc., 98, 2818 (1977)); Jacobi, et al., J. Am. Chem. Soc., 106 (19), 5594-5598 (1984); Fleming, et al., J. Am. Chem. Soc., 3926 (2006)).

The preferred source of site I sodium channel blocker is the neosaxitoxin produced by Proteus, Chile.

B. Alpha-2-Adrenergic Agonists and Antagonists, Ih Current Blockers and Enhancers

Compounds which may be used in combination with site I sodium channel blockers can be determined by the class effect by using other agents within the class. For example:

α2-AR agonists: Examples include, but are not limited to, tizanidine, medetomidine, xylazine, guanfacine, and guanethidine.

α2-AR antagonists: Examples include, but are not limited to, yohimbine, atipamezole, idazoxan, and mirtazapine.

Ih current blockers: ZD7288.

Ih current enhancer: forskolin.

Alpha-2-adrenergic agonists (α2-AR) are known to those skilled in the art. See, for example, The Pharmacological Basis of Therapeutics, 8th Edition, Gill, A. G., T. W. Rall, A. S. Nies, P. Taylor, editors (Pergamon Press, Co., Inc., NY, 1990). A preferred class of alpha 2 agonists consists of xylazine, flutonidine, moxonidine, tramazoline, tolonidine, piclonidine, tiamenidine, and dexmedetomidine.

In a preferred embodiment, the Alpha-2-adrenergic agonist is Dexmedetomidine (DMED), also known as (S)-4-[1-(2,3-Dimethylphenyl)ethyl]-3H-imidazole, marketed as PRECEDEX®, DEXDOR® or DEXDOMITOR®.

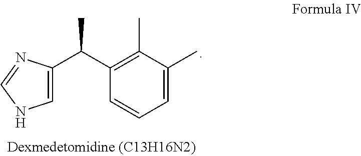

Dexmedetomidine is often used as an intravenous sedative medication in intensive care units. It has a half-life of approximately 2 hours. Side effects of Dexmedetomidine are associated with peripheral α2-receptor stimulation, resulting in hypotension (low blood pressure) and bradycardia.

DMED is also believed to block hyperpolarization-activated nonselective cation channels (HCN channels). Functional analogs and variants of Formula IV can exhibit similar effects as Dexmedetomidine in prolonging or enhancing the local analgesic effect of Site 1 Sodium Channel blockers (S1SCB).

Preferred molecules that are expected to be similar to dexmedetomidine including etomidate, histidine and fragments of dexmedetomidine, to determine the molecular determinants of dexmedetomidine effect. Marhofer, et al. Br J Anaesth. 110(3):438-42 (2013).

In certain embodiments, molecules that have a molecular structure resembling Dexmedetomidine can be used. Exemplary molecules that resemble the molecular structure of Dexmedetomidine include Etomidate and Histidine. In further embodiments, molecules that comprise fragments or oligomers of Dexmedetomidine can be used. Preferably, the fragments and/or oligomers have a similar functional activity as Dexmedetomidine.

In certain embodiments, other Alpha-2-adrenergic agonists in addition to or instead of dexmedetomidine can be utilized. Examples potentially include Tizanidine, Medetomidine, Xylazine, Guanfacine and Guanethidine, as well as functional analogs and fragments thereof.

Although described above with reference to specific compounds, one can also utilize enantiomers, stereoisomers, metabolites, derivatives and salts of the active compounds. Methods for synthesis of these compounds are known to those skilled in the art. Examples of pharmaceutically acceptable salts include, but are not limited to, mineral or organic acid salts of basic residues such as amines, and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. Conventional non-toxic salts include those derived from inorganic acids such as hydrochloric, hydrobromic, sulfuric, sulfamic, phosphoric and nitric acid, and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic and isethionic acids. The pharmaceutically acceptable salts can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed. (Mack Publishing Company, Easton, Pa., 1985, p. 1418).

A prodrug is a covalently bonded substance which releases the active parent drug in vivo. Prodrugs are prepared by modifying functional groups present in the compound in such a way that the modifications are cleaved, either in routine manipulation or in vivo, to yield the parent compound. Prodrugs include compounds wherein the hydroxy or amino group is bonded to any group that, when the prodrug is administered to a mammalian subject, cleaves to form a free hydroxyl or free amino, respectively. Examples of prodrugs include, but are not limited to, acetate, formate and benzoate derivatives of alcohol and amine functional groups.

A metabolite of the above-mentioned compounds results from biochemical processes by which living cells interact with the active parent drug or other formulas or compounds in vivo. Metabolites include products or intermediates from any metabolic pathway.

C. Hyperpolarization-Activated Cation Current (Ih) Antagonists

Hyperpolarization-activated cation current (Ih) blockers (Ih current blockers) are a group of molecules recognized for their potent and specific blockade of Hyperpolarization-activated cyclic nucleotide-gated (HCN) channels. The current through voltage-gated HCN channels is designated as Ih (also known as If or Iq). Ih has a role in establishing the resting potential of cells and generates sensory receptor potentials as well as modulating responses of sensory neurons to other stimulus-evoked currents.

Exemplary (Ih) current blockers include the compound ZD7288 ivabradine and Guanfacine.

ZD7288

A preferred (Ih) current blocker is ZD7288 [4-(N-Ethyl-N-phenylamino)-1,2 dimethyl-6-(methylamino) pyrimidinium chloride ICI-D7288 N-Ethyl-1,6-dihydro-1,2-dimethyl-6-(methylimino)-N-phenyl-4-pyrimidinamine hydrochloride].

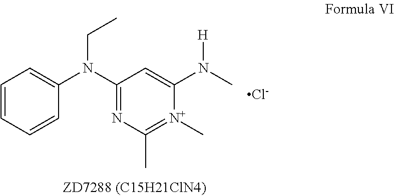 [SMILES: CCN(c1ccccc1)c1cc(NC)[n+](C)c(C)n1 ; [Cl-]]

D. Other Local Anesthetic Compounds

As used herein, the term “local anesthetic” means a drug which provides local numbness or pain relief. Classes of local anesthetics which can be utilized include the aminoacylanilide compounds such as lidocaine, prilocaine, bupivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amid compounds such as dibucaine and related local anesthetic compounds; aminoketone compounds such as falicaine, dyclonine and related local anesthetic compounds; and amino ether compounds such as pramoxine, dimethisoquin, and related local anesthetic compounds. The preferred local anesthetics are amino-amides and amino esters.

These drugs average six to ten hours of pain relief when given in different sites and for different types of surgery. For many types of surgery, it would be preferable to have durations of pain relief that last two or three days. The preferred local anesthetics for use in combination with NeoSTX are bupivacaine, ropivacaine, tetracaine and levobupivacaine. Bupivacaine is a particularly long acting and potent local anesthetic. Its other advantages include sufficient sensory anesthesia without only partial motor blockade, and wide availability.

E. Vasoconstrictors

Vasoconstrictors which are useful are those acting locally to restrict blood flow, and thereby retain the injected drugs in the region in which they are administered. This has the effect of substantially decreasing systemic toxicity. Preferred vasoconstrictors are those acting on alpha adrenergic receptors, such as epinephrine and phenylepinephrine. Other drugs and dyes vasoconstrict as a side-effect, such as bupivacaine and levobupivacaine.

F. Excipients, Delivery Vehicles and Devices

Compositions can be administered by injection, infiltration or topically. Typical carriers are saline, phosphate buffered saline, and other sterile injectable carriers. In general, pharmaceutical compositions are provided including effective amounts of an active agent, targeting moiety, and optional a delivery vehicle and optionally include pharmaceutically acceptable diluents, preservatives, solubilizers, emulsifiers, adjuvants and/or carriers. Such compositions include the diluents sterile water, buffered saline of various buffer content (e.g., Tris-HCl, acetate, phosphate), pH and ionic strength and optionally additives such as detergents and solubilizing agents (e.g., TWEEN® 20, TWEEN® 80 also referred to as polysorbate 20 or 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, and vegetable oils, such as olive oil and corn oil. The formulations may be lyophilized and redissolved/resuspended immediately before use. The formulation may be sterilized by, for example, filtration through a bacteria retaining filler, by incorporating sterilizing agents into the compositions, by irradiating the compositions, or by heating the compositions.

For application to the cornea, the formulations may be a liquid solution, suspension, or controlled release formulation such as liposomes or nanoparticles. These may be injected into the eye, the tissue surrounding the eye, or into a compartment of the eye, or topically applied to the eye or cornea. Topical administration can include application directly to exposed tissue, vasculature or to tissues or prostheses, for example, during surgery, or by direct administration to the skin. Suitable excipients for administration to the eye are known.

Formulations for administration to the mucosa (such as the conjunctiva) will typically be spray dried drug particles or liposomes, applied directly or resuspended for administration to the mucosal. Standard pharmaceutical excipients are available from any formulator. For application by the ophthalmic mucous membrane route, compositions may be formulated as eyedrops or eye ointments. These formulations may be prepared by conventional means.

In some embodiments liposomes are used as delivery vehicles. Liposomes are biodegradable, non-toxic, unilamellar or multilamellar vesicles formed from naturally occurring or synthetic phospholipids. Liposomes have an ability to entrap and retain a wide range of therapeutic agents, either in their aqueous (hydrophilic agents) or their lipid (hydrophobic) phases (Senior, Crit. Rev. Ther. Drug Carrier Sys., 3, 123-193 (1987); Lichtenberg, Methods Biochem. Anal., 33, 337-362 (1988); Gregoriadis, Subcell. Biochem., 14, 363-378 (1989); Reimer, et al., Dermatol., 195:93 (1997)). Twelve liposomal-therapeutic agent formulations have been approved by the U.S. Federal Drug Administration and an additional twenty-two were in clinical trials (Chang, et al., Scientific Rep., 1, 195 (2012)).

Liposomes are spherical vesicles composed of concentric phospholipid bilayers separated by aqueous compartments. Liposomes can adhere to and form a molecular film on cellular surfaces. Structurally, liposomes are lipid vesicles composed of concentric phospholipid bilayers which enclose an aqueous interior (Gregoriadis, et al., Int. J. Pharm., 300, 125-30 2005; Gregoriadis and Ryman, Biochem. J., 124, 58P (1971)). Hydrophobic compounds associate with the lipid phase, while hydrophilic compounds associate with the aqueous phase. Suitable methods, materials and lipids for making liposomes are known in the art. Liposome delivery vehicles are commercially available from multiple sources. The liposome may be formed from a single lipid; however, in some embodiments, the liposome is formed from a combination of more than one lipid. The lipids can be neutral, anionic or cationic at physiologic pH.

Incorporation of one or more PEGylated lipid derivatives can result in a liposome which displays polyethylene glycol chains on its surface. The resulting liposomes may possess increased stability and circulation time in vivo as compared to liposomes lacking PEG chains on their surfaces. Liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC).

The lipids can also include various natural (e.g., tissue derived L-α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids. In one embodiment, the liposomes contain a phosphaditylcholine (PC) head group In another embodiment, the liposomes contain DPPC. In a further embodiment, the liposomes contain a neutral lipid, preferably 1,2-dioleoylphosphatidylcholine (DOPC). In certain embodiments, the liposomes are generated from a single type of phospholipid. Suitable cationic lipids in the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N—(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC14-amidine, N-tert-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one embodiment, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one embodiment, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine), with cholesterol being most preferred. The molar ratio of a first phospholipid, such as sphingomyelin, to second lipid can range from about 5:1 to about 1:1 or 3:1 to about 1:1, more preferably from about 1.5:1 to about 1:1, and most preferably, the molar ratio is about 1:1.

The liposomes typically have an aqueous core. The aqueous core can contain water or a mixture of water and alcohol. Suitable alcohols include, but are not limited to, methanol, ethanol, propanol, (such as isopropanol), butanol (such as n-butanol, isobutanol, sec-butanol, tert-butanol, pentanol (such as amyl alcohol, isobutyl carbinol), hexanol (such as 1-hexanol, 2-hexanol, 3-hexanol), heptanol (such as 1-heptanol, 2-heptanol, 3-heptanol and 4-heptanol) or octanol (such as 1-octanol) or a combination thereof.

The liposomes can have either one or several aqueous compartments delineated by either one (unilamellar) or several (multilamellar) phospholipid bilayers (Sapra, et al., Curr. Drug Deliv., 2, 369-81 (2005)). Preferably, the liposomes are multilamellar. Multilamellar liposomes have more lipid bilayers for hydrophobic therapeutic agents to associate with.

In a preferred embodiment liposomes include 1,2-dioleoylphosphatidylcholine (DOPC) and 1,2-dioleoyl-sn-glycero-3-phospho-(1′-rac-glycerol) (DOPG), and optionally include cholesterol.

Controlled release polymeric devices can be made for long term release systemically following implantation of a polymeric device (rod, cylinder, film, disk) or injection (microparticles). The matrix can be in the form of microparticles such as microspheres, where active agents are dispersed within a solid polymeric matrix or microcapsules, where the core is of a different material than the polymeric shell, and the active agents are dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of active agents, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation; spray drying, solvent extraction and other methods known to those skilled in the art. Bioerodible microspheres can be prepared using any of the methods developed for making microspheres for drug delivery, for example, as described by Mathiowitz and Langer, J. Controlled Release 5, 13-22 (1987); Mathiowitz, et al., Reactive Polymers 6, 275-283 (1987); and Mathiowitz, et al., J. Appl. Polymer Sci. 35, 755-774 (1988). The formulations may also contain preservatives, pH adjusting agents, antioxidants, and isotonicity agents.

In the preferred embodiment the anesthetic is formulated in saline, or an acidic buffered solution, optionally containing a preservative. Local or sustained release carriers may be utilized.

G. Dosage Units

In preferred embodiments, the site 1 sodium channel blockers in combination with alpha-2-adrenergic agonists and optionally delivery vehicles and/or other excipients are provided in vials in an aqueous solution. Depending on the type of formulation, as outlined previously and below, the vial sizes may range from 1-15 mls, and 1-3 vials may be used for a single patient in different situations. In another embodiment, the site 1 sodium channel blockers in combination with alpha-2-adrenergic agonists, and optionally delivery vehicles and/or other excipients are provided in one or more vials, optionally lyophilized, then rehydrated and combined prior to use. For this second embodiment, preferred vial sizes could range from 5-40 mls. Dosage units for ocular formulation are based on whether or not the formulation is injectable or topically applied or instilled. The volume for intravitreal injection is typically between about 0.03 and 0.1 ml. Most formulations applied topically as drops are administered as volumes of 0.03 and 0.07 ml/drop, with a maximum typical volume of about 100 microliters. The formulations may also be provided in two units, one lyophilized and one a diluent for re-suspension of the lyophilized active.

### III. Methods of Use

Methods of prolonging the sensory and/or motor nerve blockade by site 1 sodium channel blockers (S1SCB) by combining site 1 sodium channel blockers with additional active agents, which act as anesthetic adjuvants are provided. The methods can include contacting one or more nerves with an effective amount of a site 1 sodium channel blocker in combination with one or more additional active agents to decrease or inhibit sensory activity in the nerves compared to a control. The methods can prolong the blockade of hyperpolarization-activated nonselective cation channels (HCN channels) with minimal or reduced toxicity.

In some embodiments the additional active agents stimulate a response from the adrenergic receptors. Thus, the methods can include contacting one or more nerves with an effective amount of a site 1 sodium channel blocker in combination with alpha-2-adrenergic agonists to decrease or inhibit sensory activity in the nerves compared to a control. In a preferred embodiment the additional active agent is the alpha-2-adrenergic agonist Dexmedetomidine. In some embodiments, the methods can block hyperpolarization-activated nonselective cation channels (Ih channels) and simultaneously stimulate a response from the adrenergic receptors to prolong the anesthetic effect of site 1 sodium channel blockers relative to site 1 sodium channel blockers administered alone. In some embodiments, the methods do not give rise to vasoconstriction.

In some embodiments, the methods prolong the local analgesia effect of an S1SCB in the cornea via a mechanism that is not associated vasoconstriction from α2-AR agonism and not associated with blockade of the hyperpolarization cation current (Ih) effect on block from TTX. In a particular embodiment, the methods prolong the local analgesia effect of an S1SCB in the cornea without giving rise to systemic effects, such as sedation.

Methods to prevent, reduce or inhibit sensory and/or motor function in the cornea of the eye are provided. The methods can include introducing a combination of a site 1 sodium channel blocker and an S1SCB anesthetic adjuvant into or onto the cornea or the eye. The methods can include selecting one or more S1SCB anesthetic adjuvants based on the ability to enhance or prolong the analgesic effect of a site 1 sodium channel blocker administered topically to the cornea of the eye. The enhanced or prolonged analgesic effect of a site 1 sodium channel blocker can occur in the presence or absence of effects outside of the cornea of the eye, such as systemic effects.

It may be that the ability of alpha-2-adrenergic agonists and/or hyperpolarization-activated cation current (Ih) blockers to enhance and/or prolong the duration of anesthetic block by a site 1 sodium channel blocker in the cornea is dependent upon the ability of the active agents to penetrate into and throughout the cornea and contact the target nerve(s). A preferred S1SCB anesthetic adjuvant for use in the cornea is dexmedetomidine. For example, it has been demonstrated that topical application of dexmedetomidine prolongs ocular anesthesia by tetrodotoxin, while clonidine, epinephrine (vasoconstrictor, mixed α1, α2 agonist) and phenylephrine (vasoconstrictor, pure α1) do not prolong ocular anesthesia from tetrodotoxin. Therefore, where the methods are effective to prolong ocular anesthesia from S1SCB by topical administration to the cornea of the eye, the alpha-2-adrenergic agonist is not clonidine or epinephrine. It has been established that site 1 sodium channel blockers and dexmedetomidine formulations have benign tissue reaction in the cornea.

A preferred dose volume for intravitreal injection is typically between about 0.03 and 0.1 ml. Most formulations applied topically as drops are administered as volumes of 0.03 and 0.07 ml/drop, with a maximum typical volume of about 100 microliters.

A preferred S1SCB or local anesthetic adjuvant is dexmedetomidine. Dexmedetomidine can prolong the duration of block by a site 1 sodium channel blocker or local anesthetic in a dose-dependent fashion (i.e., as the concentration of dexmedetomidine is increased, the duration of block from site 1 sodium channel blocker or local anesthetics is also increased up to a point; in the experimental animal model below, no increase in duration of block by S1SCB was obtained at a concentration of DMED of greater than 0.21 mM). Based on the dose response curve of dexmedetomidine, optimal dosing for use as a local anesthetic adjuvant has been established. In preferred embodiments, the amount of dexmedetomidine is not sufficient to induce systemic effects. In a particular embodiment, topical doses of dexmedetomidine are from 4.6 and 5.0 μg/kg body weight inclusive, administered directly to the cornea of the eye can prolong the local effect of a site 1 sodium channel blocker (S1SCB), such as tetrodotoxin in the cornea without giving rise to systemic sedation, whereas doses of dexmedetomidine greater than 4.6 and 5.0 μg/kg body weight prolong the local effect of a S1SCB in the cornea and give rise to systemic sedation. The extent and/or duration of systemic effects can vary with dosage and location of administration. For example, the extent and/or duration of sedation can correlate with the dosage of dexmedetomidine at the cornea. In a particular embodiment, the site 1 sodium channel blocker tetrodotoxin is administered to the cornea at a concentration 3.1 mM in combination with dexmedetomidine at a concentration of 0.21 mM administered as volumes of 0.03 and 0.07 ml/drop, with a maximum typical volume of about 100 microliters.

In a particular embodiment, the disclosed formulations are applied directly to the eye, topically, by injection or by instillation. Dosage units containing an amount of one or more site 1 sodium channel blockers with anesthetic adjuvants, such as the alpha-2-adrenergic agonist Dexmedetomidine to provide prolonged duration of anesthesia in the eye or ocular cavity are also provided. In other embodiments, dosage units can be prepared in an amount effective to prevent, reduce or inhibit sensory and/or motor function in the cornea of the eye.

In the preferred embodiment, the formulation is administered once, twice, three times or more than three times a day directly to the eyes of the individual in need thereof. The frequency will vary depending on the severity of symptoms. The formulation may be applied as a drop in the form of an emulsion or suspension, liposome, lotion, ointment, cream, gel, salve or powder and sustained or slow release, as well as eyelid lotion. It may also be used as an eye wash or rinse to irrigate the eye. The formulation may also be applied in a sprayable form.

In preferred embodiments the dosage administered to the eye does not result in toxicity to the eye. For example, multiple applications per day for an extended period of time do not result in damage or toxicity to the cornea. Therefore, methods of treating painful conditions of the eye by administering an effective amount of one or more site 1 sodium channel blockers with alpha-2-adrenergic agonists or hyperpolarization-activated cation current (Ih) blockers to prolong the duration of anesthesia in or around the cornea of the eye are provided.

The present invention will be further understood by reference to the following non-limiting examples.

### Example 1: Combinations of Site 1 Sodium Channel Blockers with Alpha-2-Adrenergic Agonists Prolong the Duration of Anesthesia in the Eye

The anesthetic effects of Tetrodotoxin (TTX), Dexmedetomidine (DMED), Saxitoxin (STX), Clonidine (CLD) and Proparacaine (PPC) alone was compared with the effects of Tetrodotoxin (TTX) combined with Dexmedetomidine (DMED); Tetrodotoxin (TTX) combined with Clonidine (CLON), and Dexmedetomidine (DMED) in combination with Saxitoxin (STX). Ocular anesthesia was induced by applying drops of each formulation applied to the eye. Anesthetic effect was determined using the absence of blink response to filaments of varied lengths, including 0.5 cm (Block0.5), 2 cm (Block2.0), and 6 cm (Block>6).

Materials and Methods

Materials

Tetrodotoxin (>98% purity, Abcam, Cambridge, Mass.), clonidine hydrochloride (>99% purity, Sigma-Aldrich Corp, St. Louis, Mo.) and dexmedetomidine hydrochloride (>99% purity, Tocris Bioscience, Ellisville, Mo.) formulations were prepared in 20 mM citrate solution (pH 4.5). Saxitoxin was a generous gift from Sherwood Hall (Food and Drug Administration, College Park, Md.). Proparacaine hydrochloride (Sigma-Aldrich Corp, St. Louis, Mo.) was prepared in 0.9% (w/v) saline. All drug solutions were prepared immediately before use. Thirty μL of each formulation was topically applied to the cornea. For in vitro cell viability assays, 3-(4,5-dimethylthiazol-2-yl)-5-(3-carboxymethoxyphenyl)-2-(4-sulfophenyl)-2H-tetrazolium (MTS) and phenazine methosulfate were purchased from Promega Corp (Madison, Wis.). All chemicals were used as provided by the manufacturer without additional purification.

Cell Viability Assay

Immortalized human corneal limbal epithelial (HCLE) cells were cultured in keratinocyte serum-free medium (KSFM; Invitrogen, Carlsbad, Calif.) supplemented with epidermal growth factor (EGF) and bovine pituitary extract until cells reached 50% confluence. Culture medium was then changed to 1:1 unsupplemented low-calcium DMEM and F12 Ham's nutrient mixture (Invitrogen, Carlsbad, Calif.) mixed with KSFM in a 1:1 ratio. Differentiation was induced by exposing the cells to a 1:1 mixture of DMEM/F12 medium (Invitrogen, Carlsbad, Calif.) supplemented with EGF and newborn calf serum. All cells were incubated at 37° C. in a 5% CO₂ environment. HCLE cells were then incubated in 96-well tissue culture plates with 150 μL of media containing either 3.1 mM TTX+0.21 mM dexmedetomidine, 3.1 mM TTX alone or 0.21 mM dexmedetomidine alone. All drug solutions were dissolved in 20 mM citrate buffer (pH 4.5) so that they would be in the same buffer in which TTX was dissolved. Media was prepared by adding 10× drug solution to fresh media in a 1:9 ratio so that each cell culture well contained 133.4 μL fresh media and 16.6 μL of test solution (20 mM citrate buffer with or without drugs. Solutions were filtered aseptically using a 0.2 μm syringe filter. Cellular viability was measured after 4, 8, 16 and 24 hours using the MTS colorimetric assay normalized to cells that were not exposed to drug solutions.

Animals

Six week old male Sprague-Dawley rats (Charles River Laboratories, Wilmington, Mass.) were housed in groups, in a 6 AM-6 PM light-dark cycle. Animals were cared for in accordance with protocols approved by the Animal Care and Use Committee at Boston Children's Hospital (Boston, Mass.), as well as the Guide for the Care and Use of Laboratory Animals of the US National Research Council, and the ARVO statement for the Use of Animals in Ophthalmic and Vision Research.

Application of Corneal Medications

Rats were gently restrained by wrapping them in a towel, leaving the head exposed for drug application. Animals then received local anesthetic solutions in the form of topical drops to the left eye. The right eye remained untreated to serve as a control for systemic anesthetic effect on the cornea. In studies of topical anesthetic efficacy, animals received a single dose of test solution in a volume of 30 μL. Animals in studies of healing after corneal debridement were given a dose immediately after creation of the corneal lesion and then every 12 hours until the epithelium was completely healed.

Drug Preparation

TTX formulations (Table 1) were prepared in 20 mM citrate solution (pH 4.5) with or without dexmedetomidine hydrochloride (DMED). DMED was added to TTX formulations in 3 different doses so that each 30 μL drop delivered either 50 μg/mL (0.21 mM; 5 μg/kg for a 300 g rat) DMED, 125 μg/mL (0.53 mM DMED; 12.7 μg/kg for a 300 g rat) or 250 μg/mL (1.1 mM; 25 μg/kg for a 300 g rat) DMED. These dose ranges were derived from the observation by Brummett, et al., Anesthesiology 111:1111-1119 (2009), that dexmedetomidine doses above 6 μg/kg perineurally produce undesired sedation in rats. Because drug permeation across the cornea is minimal (<10% for most drugs), DMED dose ranges of 5-25 μg/kg, which both include and exceed the 6 μg/kg perineural dose reported in the literature for rats, were selected. Systemic absorption after passage into the nasolacrimal duct can produce toxicity and so a very broad dose range was used to identify when sedation from systemic absorption may occur. TTX formulations were also prepared with the same concentrations of clonidine (0.21 mM and 1.1 mM) to facilitate comparison to dexmedetomidine.

Studies were conducted to determine if DMED would prolong ocular anesthesia from the conventional local anesthetic proparacaine (PPC). Fifteen mM PPC formulations (Table 1) were prepared in 20 mM citrate solution (pH 4.5) with or without dexmedetomidine hydrochloride. 15 mM PPC was selected because that is the concentration in broad clinical use. The same doses of DMED or CLON that were added to TTX were added to PPC solutions so that each 30 μL drop delivered either: 15 mM PPC, 15 mM PPC+0.21 mM DMED, 15 mM PPC+0.53 mM DMED, 15 mM PPC+1.1 mM DMED, 15 mM PPC+0.21 mM CLON or 15 mM PPC+1.1 mM CLON.

Assessment of Corneal Nociceptive Blockade

Corneal tactile sensitivity was tested, using a Cochet-Bonnet esthesiometer (Luneau Ophthalmologie, Chartres, France). Briefly, the esthesiometer consists of an adjustable length nylon monofilament that exerts pressure inversely proportional to its length and can be adjusted from 0.5 to 6 cm. A longer, more flexible filament length is least painful, whereas a shorter, stiffer filament length is most painful. Testing began by gently placing the tip of the fully extended monofilament perpendicularly onto the cornea and applying enough pressure to cause the filament to bend. Eyes were probed with the monofilament in this fashion three times to determine presence or absence of a blink response, starting with the filament at 6 cm. Care was taken to avoid contact with eyelashes, which could also elicit a blink. In the event of a partial blink, the cornea was probed three additional times to confirm the presence or absence of a blink response. If no blink was elicited, the filament length was reduced by 0.5 cm increments and testing repeated until a blink was elicited. Testing started 15 minutes after anesthetic drops were administered and repeated every 15 minutes until complete return to baseline. The operator testing corneal nociception was not aware of which anesthetic treatment was assigned to any given rat (i.e. she/he was “blinded”). The filament length required to elicit a blink response is a measure of the degree of analgesia. Intensity of corneal nociceptive block was described as complete for rats that failed to blink in response to a 0.5 cm filament (block_{0.5}), dense for absence of response to a 2 cm filament (block₂) and partial for absence of response to a 6 cm filament (block_{<6}). The duration of corneal block for each parameter of block intensity (block_{0.5}, block₂ and block_{<6}) was calculated as the time elapsed after application of anesthetic drops for which the blink response was absent with stimulation by a given filament length

Assessment for Sedation

Rats were assessed for sedation from systemically absorbed α₂-AR agonists immediately after application of anesthetic solutions and every 15 minutes thereafter (immediately prior to testing of corneal sensation) until ocular sensation returned to baseline. The Sedation Rating Scale for rats was used, which is a 6-point scale ranging from 0 (asleep, eyes fully closed, loss of righting reflex) to 5 (fully awake, eyes wide open, grooming, feeding and ambulating).

Corneal Epithelial Debridement Studies

Under isoflurane anesthesia, a 2 mm diameter circular defect was made in the central corneal epithelium of the right eye with a 2 mm trephine. Under a stereomicroscope, the corneal epithelium within the area demarcated by the defect was removed by gentle brushing with an Algerbrush II corneal rust ring remover fitted with a 1.0 mm burr (Ambler Surgical, Exton, Pa.), leaving the basement membrane intact. Thirty μL of drug solution were administered to the affected eye immediately after creation of the corneal lesion and then every 12 hours until the epithelium was completely healed. Fluorescein (FULGLO strips, Akron Inc, Lake Forest, Ill.) was instilled and photographs of the cornea were taken with a Nikon D90 camera fitted with a 40 mm AF-S Micro NIKKOR f/2.8 lens every 12 hours until complete re-epithelialization. An external light source with a cobalt-blue filter was used to illuminate the fluorescein filled corneal defect. Images were analyzed with Fiji (ImageJ2) software (NIH, Bethesda, Md.) to measure the wound area at each time point and calculate the rate of corneal re-epithelialization. The rate of corneal re-epithelialization was calculated by subtracting the wound area (mm²) at 24 hours to the wound area (mm²) from that immediately after debridement and dividing by 24.

Statistical Analysis

For corneal block durations and wound healing assays, data were reported as means and standard deviations of N observations and were compared using 1-way ANOVA with Bonferonni's post hoc test. Data from in vitro analysis were presented as means±standard deviations of N observations and were compared using two-way ANOVA with Bonferonni's post hoc test. All data analyses were performed using SPSS version 19 (SPSS, Inc., Chicago Ill.).

Results

Corneal Nerve Block Studies

Animals received a single 30 μL eye drop of 0.31-3.1 mM TTX, or 15 mM (0.5% w/v) proparacaine (Table 2).

The S1SCB concentrations studied were within the range of those studied by Schwartz, et al Cornea 1998; 17: 196-9; Schwartz et al., Am J Ophthalmol 1998; 125: 481-7; Schwartz, et al., Graefes Arch Clin Exp Ophthalmol 1998; 236: 790-4; Ogura et al, Eur J Pharmacol 1968; 3: 58-67; Wang et al., Cornea 2013; 32: 1040-5; Duncan, et al. Cornea 2001; 20: 639-42.

A validated rat model (Wang, et al., Cornea 2013; 32: 1040-5; Lawrenson et al., Brit. J. Ophthalmology 1993; 77: 339-43) was used to measure the tactile sensitivities of corneas after anesthetic solutions were applied.

Animals treated with 15 mM proparacaine achieved maximal corneal block (block_{0.5}) for 15 minutes, deep block (block₂) for 35 minutes and partial block (block_{<6}) for 54 minutes (Table 2). Animals treated with TTX alone demonstrated concentration-dependent corneal analgesia (Table 2). Only the highest TTX concentration (3.1 mM) yielded block_{0.5} of any duration, but only in 5 of 14 animals, whereas block_{0.5} occurred in all animals treated with 15 mM proparacaine (p=0.013, chi-square test). That concentration of TTX (3.1 mM) yielded an average block_{<6} of 86 minutes compared to 54 minutes for 15 mM proparacaine, although this difference was not statistically significant (p=0.136). In the range 0.8-2.3 mM, TTX produced block_{<6} durations comparable to those from 15 mM proparacaine (p>0.05 for all comparisons).

To test the hypothesis that dexmedetomidine would prolong the duration of corneal block from TTX, solutions of TTX alone or in combination with 0.21 mM dexmedetomidine were administered and compared the resulting corneal nerve block durations. The dexmedetomidine concentration was two to five-fold greater than that which has been studied in peripheral nerve, yet 25-50 times less than what has been studied for topical application to the cornea. Dexmedetomidine alone did not produce any degree of corneal anesthesia, but enhanced the effect of TTX and STX for all three measures of corneal block intensity (Table 1). For example, 3.1 mM TTX produced block_{0.5} for 5 minutes, block₂ for 33 minutes and block_{<6} for 86 minutes, compared to 37 minutes, 77 minutes and 153 minutes respectively for 3.1 mM TTX+0.21 mM dexmedetomidine (p<0.001 for all comparisons; these represented 2-7-fold prolongations of nerve blockade from TTX). Corneal block durations for the drug combination increased in proportion to TTX concentration.

Block_{0.5} and block₂ durations from 1.6-2.3 mM TTX co-administered with dexmedetomidine were not statistically significantly different from the same measures for proparacaine alone, but block_{<6} durations were more than twice as long as for proparacaine (p<0.05 for all block_{<6} comparisons). In contrast, 3.1 mM TTX+0.21 mM dexmedetomidine produced block_{0.5} for 37 min, block₂ for 77 min and block_{<6} for 153 minutes, which were 2-3 times longer than the corresponding blocks from proparacaine (p=0.316, 0.001 and <0.001 respectively).

To determine whether block prolongation by dexmedetomidine applies to other S1SCBs, topical formulations of 3.1 mM saxitoxin alone or in combination with 0.21 mM dexmedetomidine were administered. The durations of block_{0.5}, block₂, and block_{<6} for saxitoxin were similar to those for TTX (p=1.000 for all comparisons) and those for 3.1 mM saxitoxin+0.21 dexmedetomidine were similar to those for 3.1 mM TTX+0.21 mM dexmedetomidine (p=1.000 for all comparisons).

To determine whether block prolongation by dexmedetomidine applies to conventional local anesthetics, topical formulations of 15 mM proparacaine (PPC) alone or in combination with either 0.21 mM or 1.1 mM dexmedetomidine were administered. The durations of block_{0.5} and block₂ from 15 mM PPC+DMED (0.21 or 1.1 mM) trended toward longer duration than that of 15 mM PPC alone, but without statistical significance (P>0.05 for both comparisons). Block_{<6} for 15 mM PPC+DMED (0.21 or 1.1 mM) was longer than that of 15 mM PPC alone (P<0.05).

To determine whether other α₂-adrenergic receptor agonists would enhance corneal anesthesia from TTX in a manner similar to dexmedetomidine, topical formulations of 3.1 mM TTX+0.21 mM clonidine were administered. Surprisingly, given the effect of dexmedetomidine, the durations of block_{0.5}, block₂, and block_{<6} from 3.1 mM TTX+0.21 mM clonidine were similar to those from TTX alone (p=1.000 for all comparisons).

The blink response in the contralateral (untreated) eye was used as a measure of the degree of systemically distributed anesthetic drug. Sensory deficits were not detected in contralateral eyes for any drug formulations. The Sedation Rating Scale for rats was used to measure sedation from clonidine or dexmedetomidine. All groups, regardless of treatment, received scores of 5±0 (i.e. no sedation was observed).

Results are shown in Table 2:

TABLE 2
Effect of agents on the duration of corneal anesthesia.

| TTX | STX | DMED | CLON | PPC | Block_{0.5} | Block₂ | Block_{<6} | |
|---|---|---|---|---|---|---|---|---|
| (mM) | (mM) | (mM) | (mM) | (mM) | (min) | (min) | (min) | N |
| — | — | — | — | 15 | 18 ± 6 | 35 ± 10 | 54 ± 8 | 10 |
| — | — | 0.21 | — | 15 | 30 ± 15 | 54 ± 17* | 117 ± 16* | 5 |
| — | — | 1.1 | — | 15 | 27 ± 7 | 51 ± 8 | 84 ± 8* | 5 |
| — | — | — | 0.21 | 15 | 15 ± 11 | 27 ± 7 | 63 ± 13 | 5 |
| — | — | — | 1.1 | 15 | 18 ± 7 | 21 ± 8 | 51 ± 17 | 5 |
| 0.31 | — | — | — | — | 0 ± 0* | 4 ± 8 | 30 ± 12 | 5 |
| 0.8 | — | — | — | — | 0 ± 0* | 4 ± 8 | 41 ± 14 | 5 |
| 1.6 | — | — | — | — | 0 ± 0* | 23 ± 26 | 56 ± 23 | 5 |
| 2.3 | — | — | — | — | 0 ± 0* | 19 ± 23 | 75 ± 27 | 5 |
| 3.1 | — | — | — | — | 5 ± 7* | 33 ± 21 | 86 ± 21 | 14 |
| — | — | 0.21 | — | — | 0 ± 0* | 0 ± 0* | 0 ± 0* | 5 |
| 0.31 | — | 0.21 | — | — | 0 ± 0 | 23 ± 26 | 75 ± 12^{†} | 4 |
| 0.8 | — | 0.21 | — | — | 11 ± 23^{†} | 49 ± 19 | 98 ± 9^{†} | 4 |
| 1.6 | — | 0.21 | — | — | 45 ± 44^{†} | 68 ± 51 | 116 ± 26*^{†} | 4 |
| 2.3 | — | 0.21 | — | — | 41 ± 28^{†} | 71 ± 19^{†} | 113 ± 15* | 4 |
| 3.1 | — | 0.21 | — | — | 37 ± 19^{†‡} | 77 ± 21*^{†‡} | 153 ± 38*^{†‡} | 18 |
| 3.1 | — | 0.53 | — | — | 51 ± 42 | 90 ± 51 | 144 ± 60*^{†‡} | 5 |
| 3.1 | — | 1.1 | — | — | 54 ± 54 | 99 ± 54 | 144 ± 53*^{†‡} | 5 |
| 3.1 | — | — | 0.21 | — | 6 ± 8 | 42 ± 13 | 93 ± 13 | 5 |
| 3.1 | | | 1.1 | | 12 ± 7 | 27 ± 7 | 90 ± 11 | 5 |
| — | 3.1 | — | — | — | 0 ± 0 | 42 ± 20 | 84 ± 17 | 5 |
| — | 3.1 | 0.21 | — | — | 33 ± 16^{§} | 69 ± 41^{§} | 132 ± 34*^{§} | 5 |

Data are means ± standard deviations and compared by a 1-way ANOVA with Bonferonni's post hoc test.
p < 0.05 is considered statistically significant
*p < 0.05 when compared to 15 mM PPC
^{†}p < 0.05 when compared to equivalent TTX concentration without DMED or CLON
^{‡}p < 0.05 when compared to 3.1 mM TTX + 0.21 mM CLON
^{§}p < 0.05 when compared to 3.1 mM STX
TTX: tetrodotoxin,
STX: saxitoxin,
DMED: dexmedetomidine,
CLON: clonidine,
PPC: proparacaine
Block_{0.5} (median duration of time without blink response to a filament length of 0.5 cm)
Block₂ (median duration of time without blink response to a filament length of 2 cm)
Block_{<6} (median duration of time without blink response to any filament length less than 6 cm)

In Vitro Cytotoxicity Studies

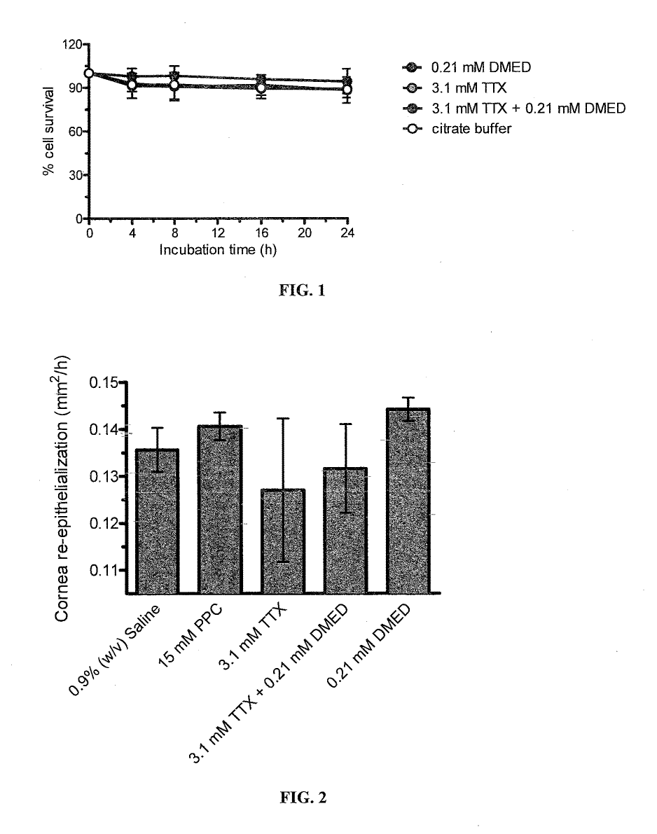

To determine the cytotoxicity of dexmedetomidine and TTX, human corneal limbal epithelial (HCLE) cells were incubated with media containing 0.21 mM dexmedetomidine, 3.1 mM TTX or 0.21 mM dexmedetomidine+3.1 mM TTX (FIG. 1). Cell viability was measured by the MTS assay over a 24 h period. HCLE cell survival was not reduced compared to cells that were not exposed to drug (p>0.05 at all time points).

Topical Anesthetic Effect on the Rate of Corneal Healing

Proparacaine and TTX+proparacaine formulations have been reported to delay corneal wound healing. To assess whether the combination of TTX+dexmedetomidine alters corneal healing, the rate of corneal re-epithelialization following debridement of 2 mm² of the corneal epithelium was measured. Drug solutions were applied to the cornea after the debridement procedure and then every 12 hours thereafter until the epithelium was completely healed. In total, three doses of drug solution were applied to animals in each group. Thirty minutes prior to applying drug solutions, the size of the corneal defect was measured as follows. Fluorescein was instilled, eyes were illuminated with an external light source using a cobalt-blue filter, and photographs were taken for digital analysis to measure the size of the defect. The rate of re-epithelialization was not decreased in any treatment group when compared to the saline control (FIG. 2; p>0.05 for all comparisons, n=5). All defects were healed by 36 hours.

The α₂-AR agonist dexmedetomidine prolonged corneal analgesia from two different S1SCBs, TTX and STX, and those combinations yielded durations of corneal analgesia 2-3 times longer than that of the widely utilized ocular anesthetic proparacaine. The durations of block achieved by co-administration of S1SCBs and dexmedetomidine, and the apparent lack of corneal toxicity of that combination indicate that such formulations may be useful treatments for acute corneal pain from a variety of conditions, including corneal abrasions or procedures such as excimer laser keratectomy and photorefractive keratectomy.

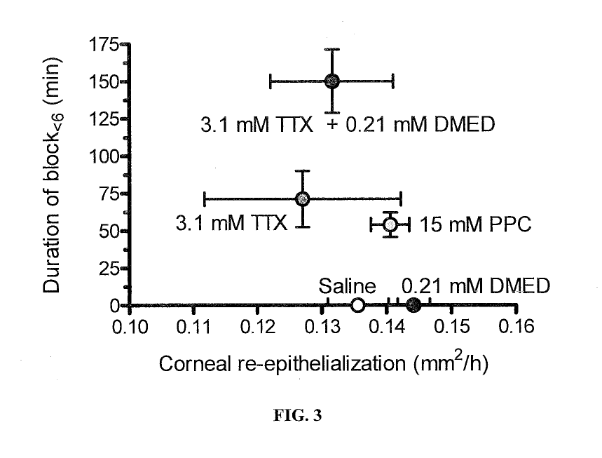

Given frequently, conventional local anesthetics are believed to produce severe corneal injury. Here, there was no apparent toxic effect of the TTX+dexmedetomidine combination in vitro or in vivo, even with prolonged or repeated administration. Also, there was no relation between the duration of nerve blockade and the rate of corneal healing (FIG. 3). It is possible that an adverse effect on healing might be seen in a more severe model of injury, or in a different injury model. These data suggest that it might be possible to use formulations of this kind for repeated and sustained corneal analgesia.

The high concentrations of TTX (and hence doses) used here (≦30 μg, compared to ≦5 μg used in peripheral nerve in rats (Padera, et al., Muscle & nerve 2006; 34: 747-53; Kohane, et al., Reg Anesth Pain Med 2001; 26: 239-45; Kohane et al. Anesthesiology 1998; 89: 119-31) raises the issue of potential systemic toxicity if absorbed into a wound or after passage into the nasolacrimal duct. Others have reported systemic toxicity in animals from topically applied TTX at concentrations much higher than were used here. No analgesia was observed in the contralateral eyes of animals, which would have suggested systemic toxicity. Systemic toxicity would become increasingly unlikely when used in larger species, including man, because the S1SCB concentration and drop volume required for local effect would be similar to that used here (30 μL), while the volume of distribution, which determines systemic toxicity, would be 200-300 times larger. Moreover, dexmedetomidine could be used to decrease the amount of S1SCB necessary to achieve a given duration of block: 0.31 mM TTX with dexmedetomidine produced similar block durations to that from 3.1 mM TTX alone (p=1.000 for all comparisons).

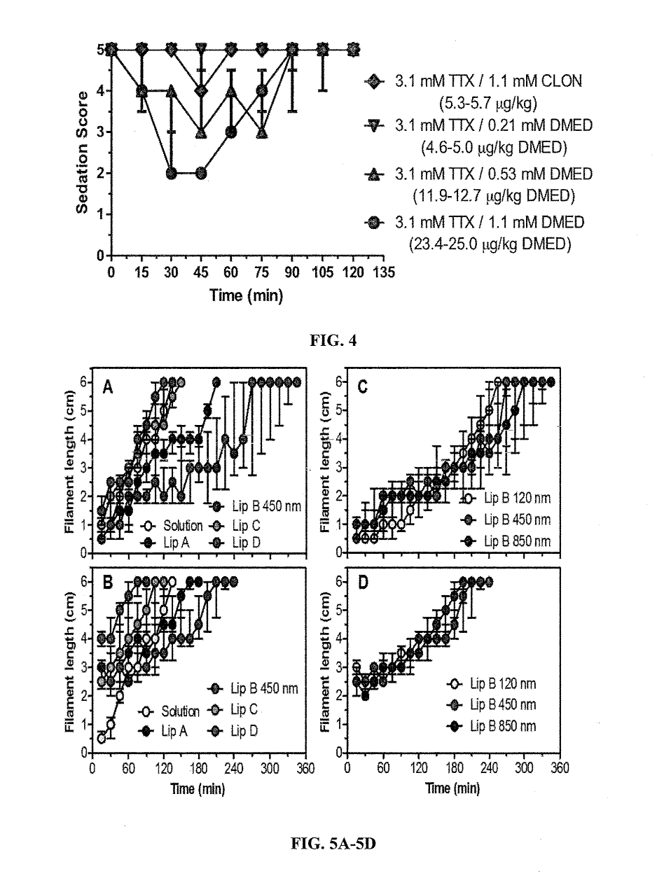

There were dissimilarities between the pharmacology on the cornea and at the sciatic nerve. Dexmedetomidine enhanced corneal block, while clonidine did not, even at concentrations much higher than those at which the latter markedly prolonged sciatic nerve blockade with TTX. This may be due to unexplored differences in drug permeability between peripheral nerve sheaths and the cornea. (In that regard, note also the large difference in the TTX concentrations required for effect in peripheral nerve and on the cornea.) Differences between clonidine and dexmedetomidine could also be attributable to differences in α-AR subtype specificity. Clonidine, which binds both α₁ and α₂-ARs, is 8 times less selective for the α₂-AR than dexmedetomidine. In addition, the α₂-AR has been divided into three pharmacologic subtypes—α_{2A}, α_{2B}, and α_{2C}—of which the α_{2A} and α_{2C} subtypes are expressed on the corneal epithelium. While dexmedetomidine and clonidine both act on all three AR subtypes, dexmedetomidine is a more potent agonist. It may be that at higher concentrations clonidine would also potentiate corneal block from S1SCBs. However, if that were the case, one would have expected the concentration of clonidine used here to have some effect since the concentrations of clonidine (and dexmedetomidine) studied here were two orders of magnitude greater than effective concentrations in other peripheral nerves (0.21 mM here compared to 2.7 μM for dexmedetomidine and 10 μM for clonidine in peripheral nerve). FIG. 4 shows similar results for the combination of DMED with a local anesthetic in prolonging the local anesthetic effect on the cornea.

The lack of effect by clonidine suggests that dexedetomidine's effect on TTX at the cornea may not be due to local α₂-AR agonist activity and its consequences. Vasoconstriction has been invoked to explain α₂-AR agonists' prolongation of peripheral nerve blockade by both S1SCBs and conventional local anesthetics. It is not clear that vasoconstriction would play a major role in drug clearance from the ocular surface since the cornea is avascular, and there are other mechanisms of elimination (tearing, drainage) that would not be affected by vasoconstriction. Moreover, the effects of clonidine and dexmedetomidine on peripheral nerve blockade by conventional local anesthetics are reported to be due to blockade of the current (Iₕ) produced by hyperpolarization-activated cation channels, and not effects on α-adrenergic receptors. Hyperpolarization-activated cation channels are expressed throughout the nervous system. Clonidine's lack of effect on the duration of corneal analgesia from TTX raises the possibility that Iₕ blockade does not play a role in dexmedetomidine's prolongation of analgesia from TTX in the cornea.

In conclusion, dexmedetomidine greatly enhances the analgesic effect of S1SCBs and conventional local anesthetics on the cornea, and dexmedetomidine-S1SCB combinations provide better corneal anesthesia than the commonly used corneal anesthetic proparacaine. Dexmedetomidine-S1SCB combinations do not cause corneal toxicity, even after repeated administration for up to 36 hours. Dexmedetomidine-S1SCB combinations may present an appealing analgesic option for corneal procedures and acute corneal pain.

### Example 2. DMED Prolongs Corneal Sensory Blockade by S1SCBs

Materials and Methods

Corneal sensory experiments were performed as in Example 1.

The solutions were prepared as in Example 1.

Results

Dexmedetomidine (DMED) prolonged corneal sensory blockade by S1SCBs (Tables 3 and 4). The findings indicate two possible mechanisms by which DMED prolongs block: (1) blockade of the Ih current and (2) agonism of the alpha-2 adrenergic receptor. This was consistent with the known mechanisms of DMED (and clonidine) at other (non-corneal) peripheral nerves. However, this result was unexpected given the observation that clonidine (an Ih current blocker and alpha-2 adrenergic receptor agonist in the same class as DMED) at any concentration failed to prolong corneal block. Because clonidine acts in a fashion similar to DMED at other peripheral nerves and clonidine is in the same class as DMED, it too should have prolonged corneal block duration. Therefore, this indicates that there is perhaps an as yet undetermined mechanism by which DMED acts on the cornea.

Another unexpected observation was that phenylephrine at low concentrations (but not high concentrations) prolonged the duration of corneal block from TTX (Table 4). This was unexpected because phenylephrine is a vasoconstrictor and the corneal surface does not have a vascular supply that should respond to vasoconstriction, and it prolonged block at low concentrations but not at higher concentration. This finding indicates that phenylephrine itself may be a potent corneal anesthetic adjuvant. These findings were unexpected because: (1) epinephrine also has potent agonist effects on the alpha 1 receptor and yet failed to prolong corneal block and (2) prazosin (an alpha 1 antagonist that functions in a manner opposite to phenylephrine) did not shorten the duration of corneal block from TTX. Our findings suggest that there may be an as yet undetermined mechanism of action by which phenylephrine acts on the cornea.

TABLE 3
A comparison of the effects of Ih current blocker (ZD7288) and
enhancer (Forskolin) on the duration of corneal block by TTX.

| TTX | ZD7288 | Forskolin | Block0.5 | Block2 | Block < 6 | |
|---|---|---|---|---|---|---|
| (mM) | (mM) | (μM) | (min) | (min) | (min) | N |
| 3.1 | 0.56 | — | 6 ± 13.4 | 54 ± 20.1 | 159 ± 25.1 | 5 |
| 3.1 | 5.6 | — | 9 ± 8.2 | 51 ± 22.7 | 171 ± 27.2 * | 5 |
| 3.1 | 56 | — | 63 ± 16.4 * | 108 ± 16.4 * | 222 ± 19.2 * | 5 |
| 3.1 | — | 76.8 | 3 ± 6.7 | 39 ± 13.4 | 120 ± 18.3 | 5 |
| 3.1 | — | 768 | 0 ± 0 | 30 ± 15 | 18 ± 16.4 | 5 |
| 3.1 | — | 1535 | 0 ± 0 | 123 ± 67 * | 108 ± 12.5 | 5 |
| 3.1 | — | — | 12 ± 12.5 | 39 ± 22.7 | 117 ± 19.5 | 5 |

Data are means ± SDs and compared by a one-way ANOVA with Bonferonni's post hoc test.
TTX, tetrodotoxin.
* P < 0.05 when compared with 3.1 mM TTX.

The data in Table 3 demonstrated that Ih current blockade (ZD7288) prolongs the duration of corneal block from TTX. Conversely, Ih current enhancement reduced the duration of corneal blockade by TTX. From these observations it was concluded that dexmedetomidine (an Ih current blocking agent) prolonged the duration of corneal block via blockade of the Ih channel.

The data in Table 4 demonstrated that (1) alpha-2 antagonists reduced the duration of corneal blockade via TTX and (2) the alpha-1 agonist phenylephrine greatly prolonged the duration of corneal block by TTX.

TABLE 4
A comparison of the effects of adrenergic receptor agonists and antagonists on the duration of corneal block from TTX.

| | Epinephrine | | | | | | | | |
|---|---|---|---|---|---|---|---|---|---|
| | (μM) | Phenylephrine | Prazosin | Yohimbine | Idazoxan | | | | |
| TTX | α1, α2, | (mM) | (μM) | (μM) | (μM) | Block0.5 | Block2 | Block < 6 | |
| (mM) | β1, β2 agonist | α1 agonist | α1 antag. | α2 antag. | α2 antag. | (min) | (min) | (min) | N |
| 3.1 | 0.55 | — | — | — | — | 0 ± 0 | 21 ± 17.1 | 111 ± 8.21 | 5 |
| 3.1 | 55 | — | — | — | — | 0 ± 0 | 27 ± 12.5 | 27 ± 22.2 | 5 |
| 3.1 | 110 | — | — | — | — | 0 ± 0 | 105 ± 10.6 | 132 ± 19.5 | 5 |
| 3.1 | — | 5.98 | — | — | — | 18 ± 16.4 | 63 ± 22.2 | 129 ± 8.2 | 5 |
| 3.1 | — | 59.8 | — | — | — | 15 ± 15 | 66 ± 20.1 | 93 ± 19.5 | 5 |
| 3.1 | — | 119.6 | — | — | — | 129 ± 8.2 | 141 ± 22.7 * | 168 ± 24.6 * | 5 |
| 3.1 | — | — | 23.82 | — | — | 0 ± 0 | 27 ± 6.7 | 111 ± 13.4 | 5 |
| 3.1 | — | — | 238.2 | — | — | 3 ± 6.7 | 42 ± 12.5 | 126 ± 17 | 5 |
| 3.1 | — | — | 476.4 | — | — | 6 ± 8.2 | 39 ± 17.1 | 123 ± 29.2 | 5 |
| 3.1 | — | — | — | 6.23 | — | 0 ± 0 * | 27 ± 12.5 | 105 ± 10.6 | 5 |
| 3.1 | — | — | — | 62.3 | — | 0 ± 0 * | 21 ± 17.1 | 111 ± 8.2 | 5 |
| 3.1 | — | — | — | 124.6 | — | 0 ± 0 * | 27 ± 22.2 | 126 ± 8.2 | 5 |
| 3.1 | — | — | — | — | 6.23 | 2 ± 4.4 | 9 ± 8.2 * | 66 ± 48.1 | 5 |
| 3.1 | — | — | — | — | 62.3 | 0 ± 0 | 3 ± 6.7 * | 63 ± 37.3 | 5 |
| 3.1 | — | — | — | — | 124.6 | 0 ± 0 | 3 ± 6.7 * | 60 ± 10.6 | 5 |
| 3.1 | — | — | — | — | — | 12 ± 12.5 | 39 ± 22.7 | 117 ± 19.5 | 5 |

Data are means 6 SDs and compared by a one-way ANOVA with Bonferonni's post hoc test.
TTX, tetrodotoxin.
* P < 0.05 when compared with 3.1 mM TTX.

### Example 3: Liposomal Formulations of Combinations of Site 1 Sodium Channel Blockers with Alpha-2-Adrenergic-Agonists

Materials and Methods

Controlled release formulations releasing site 1 sodium channel blockers and alpha-2-adrenergic agonists for extended periods of time were developed by encapsulating dexmedetomidine (DMED) and tetrodotoxin into Liposomes, using the parameters demonstrated in Table 5.

TABLE 5
Characterization of liposomal formulations containing DMED and TTX. Liposomes
consisted of 1,2-dioleoyl-sn-glycero-3-phosphocholine (PC), 1,2-dioleoyl-
sn-glycero-3-phospho-(1′-rac-glycerol) (PG) and cholesterol. The molar
ratio between total lipid (PC + PG) and cholesterol was 4:2.

| | | | | | Encapsulation | |
|---|---|---|---|---|---|---|
| | | | | | efficiency/% | |
| | PC/PG | Size/nm | Polydispersity | Zeta potential/mV | DMED | TTX |
| LIP A | 16:0 | 2010 | 0.617 ± 0.057 | −0.75 ± 0.62 | 58.3 ± 7.33 | 26.3 ± 4.75 |
| Lip B (450 nm) | 15:1 | 446 | 0.330 ± 0.011 | −11.7 ± 1.14 | 64.5 ± 5.18 | 44.2 ± 3.54 |
| Lip C | 14:2 | 502 | 0.332 ± 0.012 | −24.8 ± 0.696 | 71.9 ± 3.18 | 41.7 ± 3.06 |
| Lip D | 12:4 | 449 | 0.317 ± 0.017 | −28.9 ± 1.04 | 73.0 ± 6.16 | 51.9 ± 3.09 |
| Lip B (120 nm) | 15:1 | 117 | 0.102 ± 0.009 | −8.21 ± 2.81 | 21.9 ± 4.27 | 10.6 ± 3.71 |
| Lip B (850 nm) | 15:1 | 856 | 0.346 ± 0.023 | −7.72 ± 1.13 | 77.3 ± 6.91 | 37.6 ± 3.14 |

In all liposomes formulations, DMED free base was co-dissolved with lipids before forming thin film, which was further hydrated with TTX solution in phosphate buffered saline (PBS). In all formulations the concentrations of TTX and DMED were respective 1 mg/mL and 100 μg/mL. The TTX and DMED concentrations in liposomes were calculated based on encapsulation efficiencies.

The formulations were applied to rat corneas and tested for extent of pain relief using filaments of decreasing length, as described above.

Results

The encapsulation of Dexmedetomidine (DMED) and Tetrodotoxin (TTX) into liposomes prolonged anesthetic effect and reduced toxicity, as demonstrated in FIG. 5A-5D. FIGS. 5A and 5C demonstrate the effects of TTX/DMED solution and liposomes without dialysis (with unencapsulated free drug in the formulations) on ocular anesthesia. FIGS. 5B and 5D display the effects of TTX/DMED solution and liposomes after dialysis (without unencapsulated free drug in the formulations) on ocular anesthesia. The results demonstrate differences due to composition (FIGS. 5A, B) as well as size (FIGS. 5C, 5D) of the liposomes.

A lipid composition of 15:1 PC:PG was determined as having the optimal effect in prolonging anesthesia relative to control (see FIGS. 5A and 5C)

Those skilled in the art will recognize, or be able to ascertain using no more than routine experimentation, many equivalents to the specific embodiments of the invention described herein. Such equivalents are intended to be encompassed by the following claims.

We claim:

1. An ophthalmologic composition wherein the active agents consist of an effective amount of
one or more site 1 sodium channel blockers; and
one or more agents having hyperpolarization-activated cyclic nucleotide-gated channel antagonistic activity and/or an alpha-2 adrenergic agonist,
to reduce, decrease, or inhibit sensory and/or motor function in a nerve compared to a control without causing a systemic effect,
in an ophthalmologically acceptable carrier for application onto or into the eye or a tissue adjacent thereto.

2. The composition of claim 1, wherein the one or more site 1 sodium channel blockers are selected from the group consisting of tetrodotoxin; saxitoxin; decarbamoyl saxitoxin; Neosaxitoxin; gonyautoxins; and conotoxins.

3. The ophthalmologic composition of claim 1, wherein one site 1 sodium channel blocker is tetrodotoxin.

4. The ophthalmologic composition of claim 1 wherein the composition comprises an alpha-2-adrenergic agonist and/or a vasoconstrictor

5. The ophthalmologic composition of claim 1, comprising dexmedetomidine.

6. The ophthalmologic composition of claim 1 in an amount effective for administration into the eye to provide nerve blockade of prolonged duration on or in the eye or ocular cavity.

7. The ophthalmologic composition of claim 1 formulated in liposomes.

8. A method for preventing or alleviating pain of the eye, a compartment therein, or tissue adjacent thereto, comprising administering into or onto the eye, the compartment or the tissue an effective amount of the ophthalmologic composition of claim 1 to provide nerve blockade without systemic effects.

9. The method of claim 8, wherein the amount of one or more site 1 sodium channel blockers and dexmedetomidine is not toxic to the cornea.

10. The method of claim 9, wherein the amount of one or more site 1 sodium channel blockers and dexmedetomidine does not produce a systemic effect such as sedation.

11. An ophthalmologic composition wherein the active agents consist of an effective amount of
one or more local anesthetics; and
one or more agents having hyperpolarization-activated cyclic nucleotide-gated channel antagonistic activity,
to reduce, decrease, or inhibit sensory and/or motor function in a nerve compared to a control without causing a systemic effect,
in an ophthalmologically acceptable carrier for application onto or into the eye or a tissue adjacent thereto.

12. The composition of claim 11 wherein the agent having hyperpolarization-activated cyclic nucleotide-gated channel antagonistic activity is dexmedetomidine.

13. The composition of claim 11 wherein the local anesthetic is selected from the group consisting of aminoacylanilide compounds such as lidocaine, prilocaine, bupivacaine, mepivacaine and related local anesthetic compounds having various substituents on the ring system or amine nitrogen; the aminoalkyl benzoate compounds, such as procaine, chloroprocaine, propoxycaine, hexylcaine, tetracaine, cyclomethycaine, benoxinate, butacaine, proparacaine, and related local anesthetic compounds; cocaine and related local anesthetic compounds; amino carbonate compounds such as diperodon and related local anesthetic compounds; N-phenylamidine compounds such as phenacaine and related anesthetic compounds; N-aminoalkyl amid compounds such as dibucaine and related local anesthetic compounds; aminoketone compounds such as falicaine, dyclonine and related local anesthetic compounds; and amino ether compounds such as pramoxine, dimethisoquin, and related local anesthetic compounds.

14. The composition of claim 11 comprising proparacaine in combination with dexmedetomidine.

15. The ophthalmologic composition of claim 11 formulated in liposomes.

16. The ophthalmologic composition of claim 11 comprising an alpha-2-adrenergic agonist and/or a vasoconstrictor.

17. A method for preventing or alleviating pain of the eye, a compartment therein, or tissue adjacent thereto, comprising administering into or onto the eye, the compartment or the tissue an effective amount of the ophthalmologic composition of claim 11 to provide nerve blockade without systemic effects.
